(19) United States
(12) Patent Application Publication
(10) Pub. No.: US 2014/0214063 A1
(43) Pub. Date: Jul. 31, 2014

(54) OPERATIVE METHOD FOR LUMEN

(71) Applicant: OLYMPUS MEDICAL SYSTEMS CORP., Tokyo (JP)

(72) Inventors: Manabu MIYAMOTO, Musashino-shi (JP); Kazuo BANJU, Hachioji-shi (JP); Shotaro TAKEMOTO, Tokyo (JP); Shinji TAKAHASHI, Kokubunji-shi (JP); Takayasu MIKKAICHI, Fuchu-shi (JP)

(73) Assignee: OLYMPUS MEDICAL SYSTEMS CORP., Tokyo (JP)

(21) Appl. No.: 13/755,669

(22) Filed: Jan. 31, 2013

(51) Int. Cl. A61B 17/3205

(52) U.S. Cl. CPC A61B 17/3205
USPC 606/167

(57) ABSTRACT

Full-thickness excision of tissue of a lesion in a lumen is performed through a natural orifice. Specifically, the tissue surrounding the lesion that is in a folded state is joined and severed using a linear stapler. The linear stapler includes a cutter and is inserted into the lumen. Joining and cutting is performed while organs outside of the lumen are pushed in a direction away from the lesion with the tip of the linear stapler. As a result of these steps being repeatedly performed along the periphery of the lesion, the lesion is excised.

### BACKGROUND OF THE INVENTION

1. Field of the Invention

The present invention relates to an operative method for a lumen. In particular, the present invention relates to a method for performing full-thickness resection of a lumen wall.

2. Description of the Related Art

Surgery requiring full-thickness resection of a lumen wall, such as for a gastric submucosal tumor, is generally performed by laparotomy or laparoscopic surgery. However, the demand for minimally invasive, function-preserving endoscopic surgery is high. Even compared to the minimally invasive laparoscopic surgery, endoscopic surgery achieves the following effects. In laparoscopic surgery, when the lumen is incised, there is risk that the contents of the lumen will leak into the abdominal cavity and scatter cancer cells into the abdominal cavity (dissemination). On the other hand, in endoscopic surgery, the periphery of a lesion can be jointed together, the lesion can be excised, and the excised piece can be collected from within the hollow organ. Therefore, risk of dissemination and recurrence is reduced.

However, when full-thickness excision is performed from within the hollow organ, because the conditions on the abdominal cavity side cannot be known, there is risk of damage to other organs within the abdominal cavity.

### SUMMARY

The present invention provides an operative method enabling efficient, endoscopic full-thickness excision of a wide lesion, without contact with the lesion or damage to organs outside of a lumen, such as organs on the abdominal cavity side.

According to an aspect of the present invention, an operative method performs full-thickness excision of tissue of a lesion in a lumen thorough the lumen. The operative method performs full-thickness excision of the tissue of the lesion in the lumen by a method where an instrument is inserted from a natural orifice such as the mouth or anus, i.e. performs the excision through a natural orifice. The operative method includes: a folding step of folding a lumen wall of an excision site such that an outer surface of the lumen wall faces inward, while pushing external organs outside of the lumen from inside of the lumen in a direction away from the excision site, such as to exclude the external organs outside of the lumen from the excision site; and a joining and cutting step of joining and cutting a site where processing at the folding step is performed, using a linear stapler including a cutting member, and forming a joined section in which layers of the folded tissue are joined and a cut surface of the tissue in a folded state. The lesion is excised by the joining and cutting step being repeatedly performed along the periphery of the legion.

### BRIEF DESCRIPTION OF THE DRAWING

FIG. 1A is a schematic diagram of an overview of an endoscopic system according to embodiments of the present invention;

FIG. 1B is an enlarged perspective view of an overview of a tip in FIG. 1A;

FIG. 1C is a perspective view of an example of a usage state of the tip in FIG. 1B;

FIG. 2A is a cross-sectional schematic diagram of a working principle of a linear stapler;

FIG. 2B is a planar view of a jaw of the linear stapler;

FIG. 3 is a flowchart of an example of an operative method according to a first embodiment;

FIG. 4 is an outer appearance view of an insertion state of an endoscope;

FIG. 5 is a schematic diagram of a relationship between a lesion and a procedure site;

FIG. 6A is a schematic diagram of full thickness being grasped at a grasping point;

FIG. 6B is a cross-sectional view taken along line B-B in FIG. 6A;

FIG. 6C is a schematic diagram of tissue being is drawn in by a grasping section;

FIG. 6D is a cross-sectional view taken along line D-D in FIG. 6C;

FIG. 6E is a schematic diagram of a state during joining and cutting;

FIG. 6F is a cross-sectional view taken along line F-F in FIG. 6E;

FIG. 6G is a schematic diagram of a state after joining and cutting;

FIG. 6H is a schematic diagram for describing a next grasping point;

FIG. 6I is a schematic diagram of tissue being drawn in after being grasped;

FIG. 6J is a schematic diagram of a state during joining and cutting;

FIG. 6K is a schematic diagram of the tissue including the lesion being excised;

FIG. 7 is a schematic diagram of an example of an operative method according to a second embodiment;

FIG. 8A is a schematic diagram of an example of an operative method according to a third embodiment;

FIG. 8B is a schematic diagram for describing an instance that is not according to the third embodiment;

FIG. 9 is a schematic diagram of an example of an operative method according to a fourth embodiment;

FIG. 10 is a flowchart of an example of an operative method according to a fifth embodiment; and

FIG. 11 is a schematic diagram for describing a variation example.

### DETAILED DESCRIPTION OF THE PREFERRED EMBODIMENTS

Embodiments of the present invention will hereinafter be described with reference to the drawings.

<Endoscopic System>

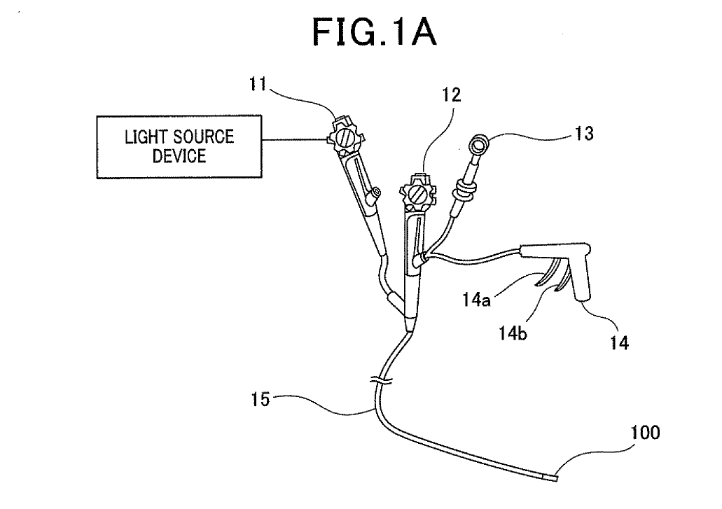

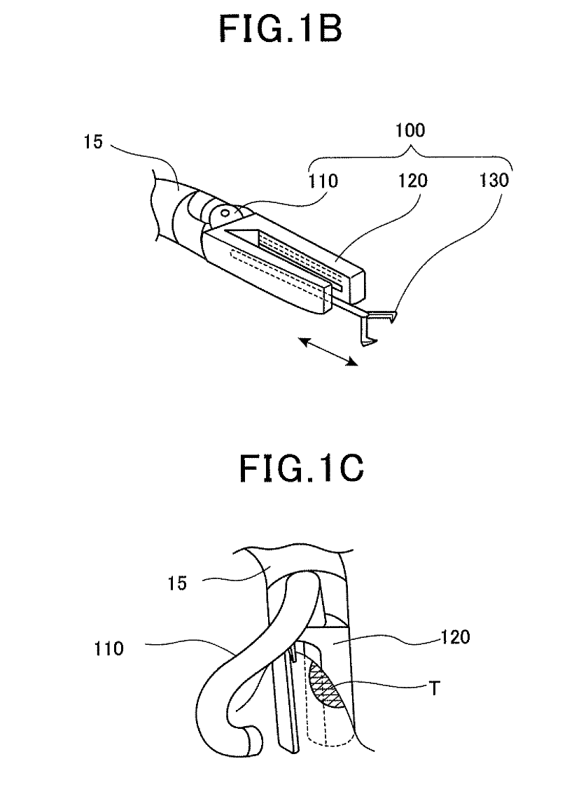

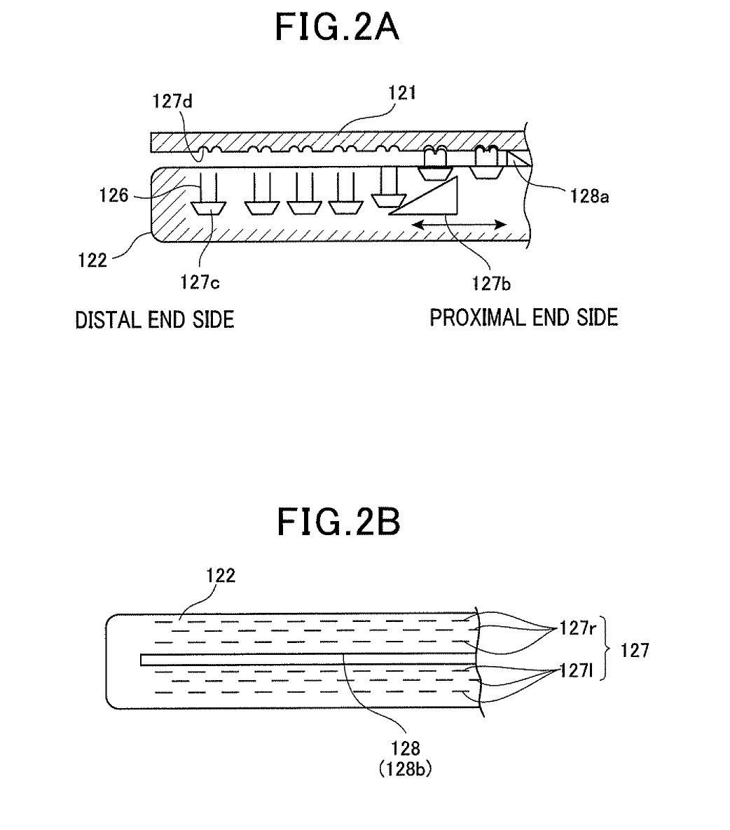

First, an endoscopic system used to perform an operative method according to the present embodiments will be described with reference to FIG. 1A to FIG. 1C, FIG. 2A, and FIG. 2B. Here, FIG. 1A is a perspective view of a portion of the endoscopic system that can be used according to the present embodiments. FIG. 1B is an enlarged perspective view of a working section of the endoscopic system. FIG. 1C is a perspective view of a usage state of an endoscope.

The endoscopic system according to the present embodiments includes a working section 100 and various operation sections 11, 12, 13, and 14, as shown in FIG. 1A. The working section 100 performs the operative method according to the present embodiments within the gastrointestinal tract. The operation sections 11, 12, 13, and 14 are used to operate the working section 100. In addition, the endoscopic system includes an insertion section 15. The insertion section 15 is inserted into the gastrointestinal tract, and connects the operation sections 11, 12, 13, and 14 with the working section 100.

The working section 100 includes an endoscope 110, a linear stapler 120 and a grasping section 130, as shown in FIG. 1B and FIG. 1C. The linear stapler 120 as an example of a stapler includes a cutting section 128 and a joining section 127. The grasping section 130 is configured to grasp the full thickness of a wall of the gastrointestinal tract.

A known configuration for capturing images of the interior of the gastrointestinal tract can be used in the endoscope 110. The endoscope 110 outputs imaging signals of the interior of the gastrointestinal tract to a monitor of a light source device. The light source device supplies light, as well as air and the like to be sent into the gastrointestinal tract. According to the present embodiments, the endoscope 110 is housed within the working section 100. In addition, the endoscope 110 is configured to move independently of the working section 100, as shown in FIG. 1C. The section which grasps the tissue, the tissue in the grasped state, and the like can be confirmed.

The linear stapler 120, described in detail hereafter, is shaped such as to extend in the axial direction of the insertion section 15.

The grasping section 130 is configured to be capable of moving along the axial direction from a distal portion to a proximal portion of the linear stapler 120. The grasping section 130 moves in the distal direction and grasps the gastrointestinal tract tissue. The grasping section 130 then retracts to the base portion (proximal end) of the linear stapler 120. As a result, the tissue is drawn into the linear stapler 120. Grasping forceps are used as the grasping section 130 according to the present embodiments. The grasping section 130 corresponds to a “pulling member” in the claims.

The operation sections 11, 12, 13, and 14 include a main operation section 12, an endoscope operation section 11, a grasping section operation section 13, and a stapler operation section 14. The main operation section 12 is operated to decide the orientation of the working section 100. The endoscope operation section 11 is operated to adjust the orientation and back-and-forth movement of the endoscope 110. The grasping section operation section 13 is operated to perform back-and-forth movement and grasping motion (grasped state and grasp release) of the grasping section 130. The stapler operation section 140 includes a first operation section 14a and a second operation section 14b. The first operation section 14a is used to perform joining and cutting operations. The second operation section 14b is used to perform opening and closing movements of the linear stapler 120.

The insertion section 15 according to the present embodiments includes a flexible tube that is capable of actively flexing. A first power transmission member, such as an angle wire, for adjusting the orientation of the working section 100, and, a second power transmission member for adjusting the orientation of the endoscope 110 are passed through the flexible tube. In addition, various components for operating the endoscope 110, the flexible tube, the linear stapler 120, and the grasping section 130 based on operations of the operation sections 11, 12, 13, and 14 are passed through the flexible tube.

FIG. 2A is a diagram schematically showing a partial cross-section of the linear stapler 120. FIG. 2B is a partial planar view of a first jaw 122 of the linear stapler 120.

As shown in FIG. 2B, the linear stapler 120 includes a joining section 127 and a cutting section 128. The joining section 127 joins, along the axial direction, the tissue that has been drawn into the linear stapler 120. The cutting section 128 cuts the tissue along the axial direction. According to the present embodiments, the cutting section 128 cuts, in the thickness direction, the tissue that is in a folded state and forms cut edges on both sides of the cutting section 128, the cut edges having overlapping cut surfaces. The joining section 127 of the linear stapler 120 forms joined sections in the overlapping tissues (i.e. layers of folded tissue) on both sides of the cutting section 128. Therefore, the joining section 127 of the linear stapler 120 is provided with staple opening rows 127r and 127l on both sides of the cutting section 128. A plurality of staples 126 are fired along the axial direction from the staple opening rows 127r and 127l. In addition, each side of the cutting section 128 is provided with a plurality of staple opening rows 127r or a plurality of staple opening rows 127l. Adjacent staple opening rows 127r are provided in positions offset from one another. Adjacent staple opening rows 127l are provided in positions offset from one another.

More specifically, as shown in FIG. 2A, the linear stapler 120 according to the present embodiments includes a first jaw 122 and a second jaw 121. The first jaw 122 fires the staples 126. The second jaw 121 receives the staples 126 that have been fired. The first jaw 122 and the second jaw 121 are connected at one end in the axial direction (hereinafter referred to as a “proximal end”) and supported by the insertion section 15. The first jaw 122 and the second jaw 121 open and close at the other end (a “distal end”). Drivers 127c and a wedge 127b are provided within the first jaw 122. The drivers 127c hold the staples 126. The wedge 127b is configured to move back and forth along the axial direction such that the wedge 127b moving in the distal end direction successively pushes out the staples 126 towards the second jaw 121 side. Each staple 126 is pushed against a recessing section 127d of the second jaw, thereby bending at the tips. The first jaw 122 includes a knife 128a that moves in coordination with the wedge 127b. As the wedge 127b advances in the distal end direction, the knife 128a advances in the distal end direction following the wedge 127b. The knife 128a cuts between the joined sections of the tissues that has been already joined.

### Operative Method According to the First Embodiment

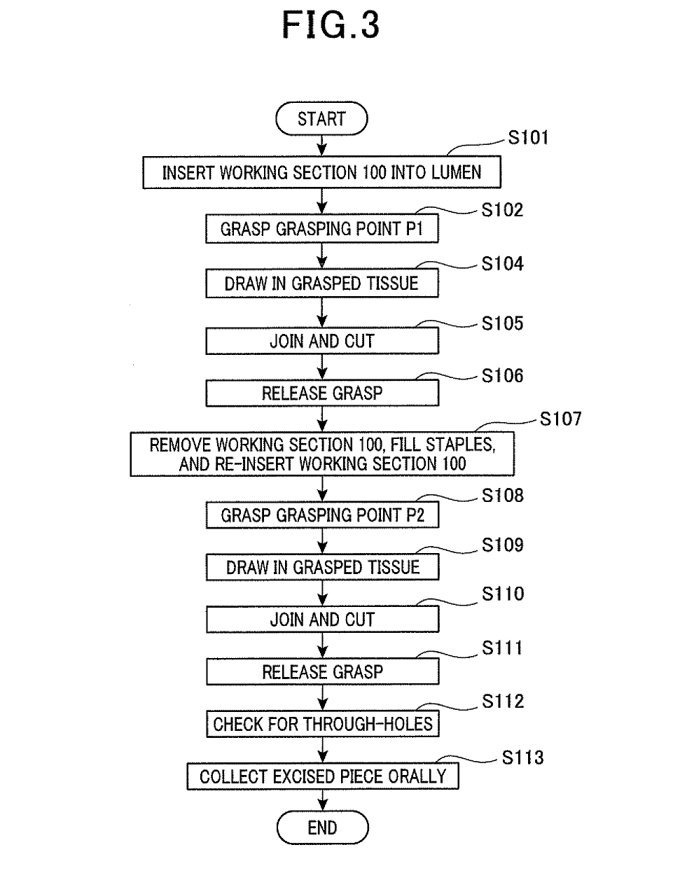

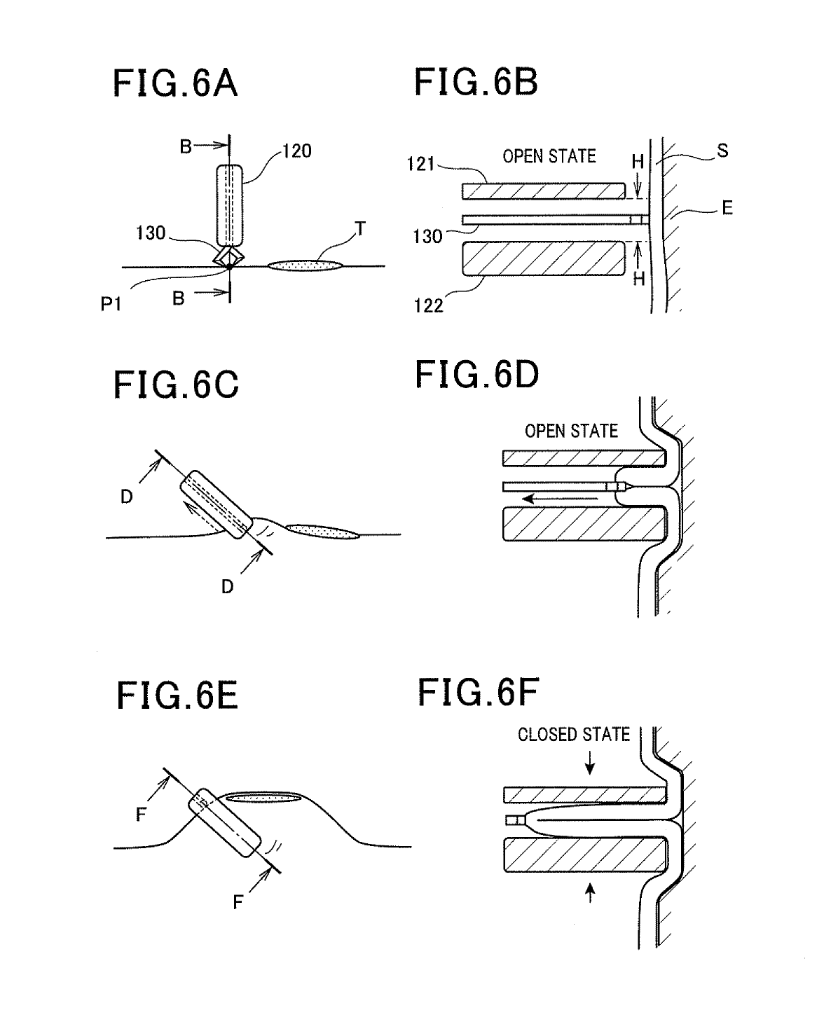

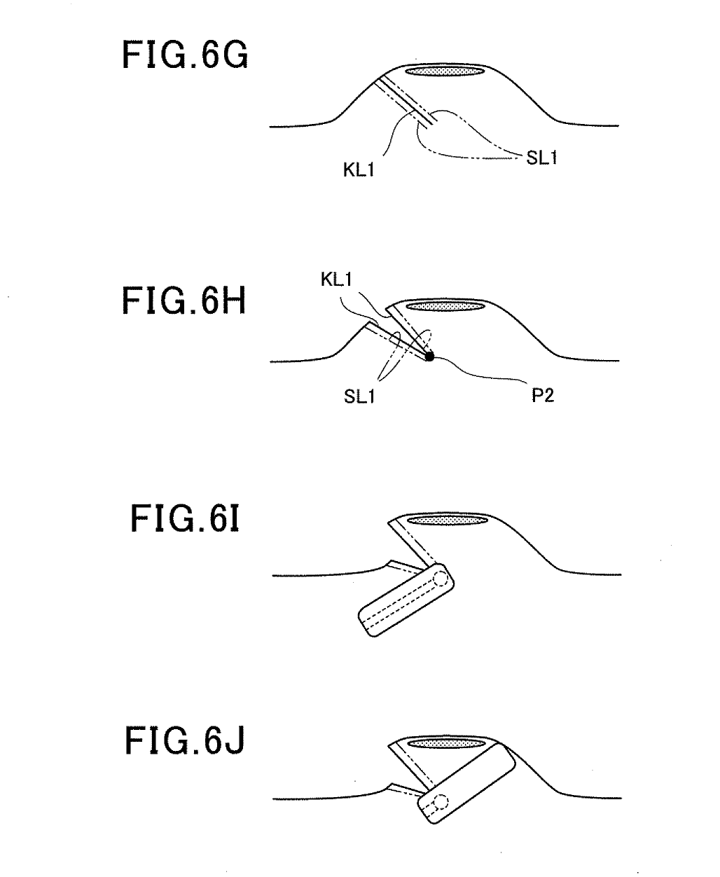

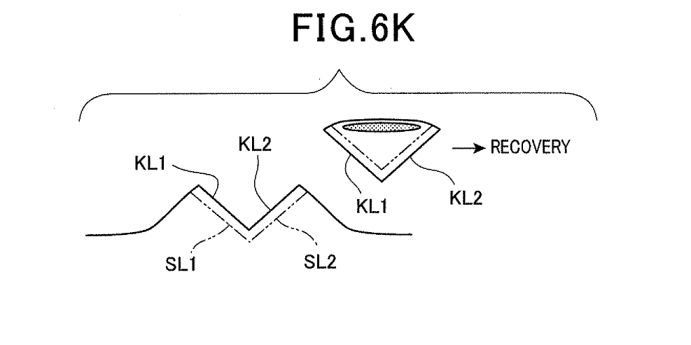

Next, an operative method according to a first embodiment will be described with reference to FIG. 3 to FIG. 5, and FIG. 6A to FIG. 6K. Here, as an example, an instance is described in which a lesion T (such as a tumor having a diameter of 20 mm) present within the gastric wall, such as that shown in FIG. 5, is excised. In this instance, the full-thickness of the gastric wall (mucosal layer, submucosal layer, muscular layer, and the like) is excised. For illustrative convenience, the lesion T is shown on the surface in the drawings. However, the present invention is not necessarily limited to instances in which the lesion is completely present on the mucosal surface. In some instances, the lesion is formed within the gastrointestinal tract tissue, such as in the submucosal layer.

FIG. 3 is a flowchart of an example of the operative method. FIG. 6A to FIG. 6K are schematic diagrams for describing the state of the lesion T in correspondence with the flowchart. For convenience, the area from the proximal end of the linear stapler 120 of the working section 100 towards the insertion section 15 side is not shown in FIG. 6A to FIG. 6K.

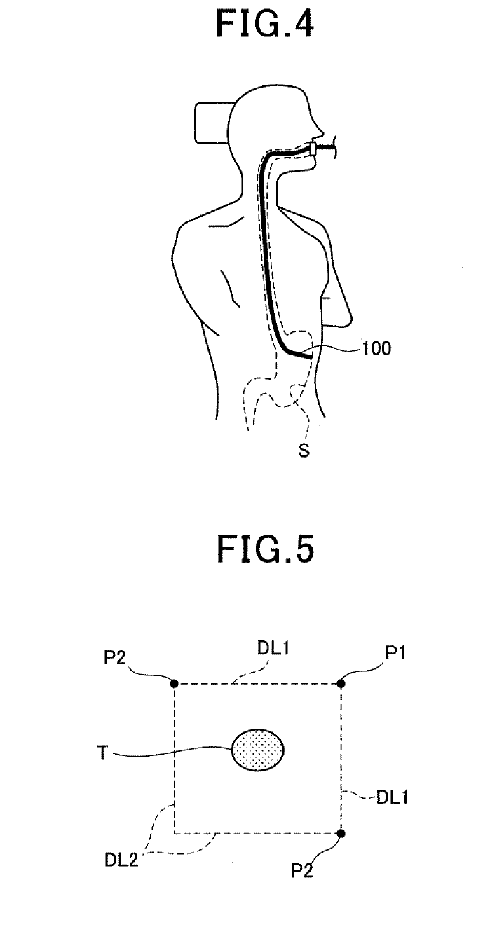

First, at Step S101, as shown in FIG. 4, the above-described working section 100 of the endoscopic system is inserted into the lumen from a natural orifice of the gastrointestinal tract (in this instance, the mouth), and positioned near the lesion T. Subsequently, air is sent into the lumen, thereby expanding the lumen. As a result, the field of view is secured.

Next, the grasping section 120 is sent out to the distal side of the linear stapler 120 and grasps a grasping point P1. The grasping point P1 is set on the gastrointestinal tract tissue surrounding the lesion T (Step S102, and FIG. 6A and FIG. 6B). As shown in FIG. 5, the grasping point P1 to be grasped by the grasping section 130 and a draw-in line DL1 are set such as to surround the periphery of the lesion T in a position that does not come into contact with the lesion T. The draw-in line DL1 indicates the direction in which the tissue is drawn into the linear stapler 120. According to the first embodiment, a stapling procedure (joining and cutting using the staple) is performed twice. Therefore, a grasping point P2 and a draw-in line DL2 for the second stapling procedure are similarly set such as to surround the periphery of the lesion T. To clarify the work site, the grasping points P1 and P2, and a portion of the draw-in lines DL1 and DL2 may be marked before the grasping operation is started. Marking can be performed, for example, by heat denaturation by a radiofrequency electrode, or by using a clip. In addition, an operation for incising the mucous membrane and the like using an incision device, such as a high-frequency knife, and exposing the muscular layer may be performed before the grasping operation.

Next, after the tissue is grasped, the grasping section 130 is drawn into the linear stapler 120 (Step S104, and FIG. 6C and FIG. 6D). Specifically, the grasping section 130 is retracted to the proximal end side of the linear stapler 120. As a result, as shown in FIG. 6C, the gastrointestinal tract tissue is drawn in by the grasping section 130, between the pair of jaws 121 and 122 forming a procedure space of the linear stapler 120. The gastrointestinal tract tissue is drawn in such that the outer surface of the gastrointestinal tract wall is folded inward. On the other hand, the external tissue E (such as the spleen, liver, and colon in this instance) outside of the gastrointestinal tract is pushed in to the direction away from the lesion T by an entry portion, with the gastrointestinal tract wall S therebetween. The entry portion forms the opening to the procedure space of the linear stapler 120. In other words, the entry portion in this instance is the tip surface of the linear stapler 120. An opening width H of the linear stapler 120, or in other words, the space between the jaws 121 and 122 at the tip in this instance, is set to a size that prevents the external tissue E from becoming caught between the gastrointestinal tract wall S that is folded. The opening width H is maintained while the tissue is being drawn into the linear stapler 120.

After the required area is drawn in along the draw-in line DL1, the jaws 121 and 122 are closed. The tissue grasped by the grasping section 120 is sandwiched between the jaws 121 and 122, and subsequently joined and cut (Step S105, and FIG. 6E and FIG. 6F). As described above, the opening width H of the jaws 121 and 122 is set. While the tissue is being pulled into the linear stapler 122, the external tissue E is pushed in the opposite direction. Therefore, the external tissue E is excluded from the cutting area (this operation is also referred to, hereinafter, as “push and displacement”). Therefore, the external tissue E is prevented from being sandwiched between the jaws 121 and 122, and therefore prevented from being joined or cut.

After joining and cutting, the jaws 121 and 122 are opened, releasing the tissue from the grasped state (Step S106). The working section 100 is temporarily removed from the body such that the linear stapler 120 can be filled with staples 126 (Step S107).

As shown in FIG. 6G, cut edges KL1 that extend along the draw-in line DU are formed in the tissue that has been released from grasp. The tissue is separated into a portion including the lesion T and a portion not including the lesion T. A joined section SL1 that extends along the cut edge KL1 is formed in each portion, such as to be further inward than the cut edge KL1. The joined sections SL 1 prevent leakage of contents within the lumen from the lumen into the abdominal cavity, as well as dissemination into the abdominal cavity as a result of leakage of the lesion T during surgery. For clarity of description, the shape of the tissue is partially retained even after the tissue is released from the grasped state by the linear stapler 120. However, in actuality, the tissue is pushed towards the outer side of the stomach by internal pressure due to air within the gastrointestinal tract, and has a slightly squashed shape.

After being filled with staples 126, the working section 100 is re-inserted into the body and sent near the lesion T. The grasping section 130 is moved forward and grasps the grasping point P2 (Step S108 and FIG. 6I). Then, while checking that the linear stapler 120 is not in contact with the lesion T, the grasping section 130 is retracted or the linear stapler 120 is moved forward. In other words, the grasping section 130 is relatively moved, and draws in the tissue along the draw-in line DL2 (Step S109 and FIG. 63). Subsequently, the jaws 121 and 122 are closed, and the tissue is joined and cut (S110). As a result, the cut edges KL2 that extend along the draw-in line DL2 are formed. The lesion T is excise from the gastrointestinal tract. In addition, the joined section SL2 is formed along the cut edge KL2 in the remaining site of the gastrointestinal tract, the joined section SL2 is continuously joined with the joining section SL1. The stapled surfaces of the first and second stapling procedures do not need to match. In other words, a line in which the end portions of the staples 126 are exposed and a line in which the connected portions of the staples 126 are exposed can be made continuous. This can be selected randomly depending on the operating circumstances.

After joining and cutting, the jaws 121 and 122 are opened, releasing the tissue from the grasped state (Step S111). Air is then sent into the lumen, and the lumen wall is checked for through-holes leading to the abdominal cavity (Step S112). To check for through-holes, visual confirmation may be performed using endoscopic images. Alternatively, whether or not air is leaking may be checked by measurement of the speed at which the lumen deflates.

Then, the excised piece including the lesion T is grasped by grasping forceps or the like that have been prepared separately. The working section 100 is removed from the body, and the excised piece is collected orally (Step S113 and FIG. 6K). At this time, the rectangular tissue portion surrounded by the draw-in lines DL1 and DL2 in FIG. 5 is collected as a folded, triangular excised piece, as shown in FIG. 6K. As a result of the excised piece being collected orally, the risk of recurrence resulting from dissemination can be reduced.

As described above, joining and cutting is repeatedly performed by the linear stapler 120 while pushing and displacing the external tissue, along the periphery of the lesion T. As a result, the lesion T is excised. Therefore, the lesion T can be excised based on its shape, without damaging the organs on the abdominal cavity side or contaminating the abdominal cavity side.

In addition, as a result of push and displacement being performed by the linear stapler 120 or by wringing at the distal end of the linear stapler 120, the push and displacement, joining, and cutting operations can be performed without requiring a larger device.

In addition, as a result of push and displacement being performed in the formation direction of the cut edges, the tissue outside of the gastrointestinal tract can be prevented from being joined and cut with the gastrointestinal tract over the entire excision site, without contact with the lesion T.

In addition, because pull (drawing in) of the tissue is also performed in the formation direction of the cut edges, the grasping areas of the tissue can be continuously set, even when the grasping area changes with each cutting procedure. In this instance, the lesion T is not grasped. In addition, the grasping section 130, the joining section 127, and the cutting section 128 do not cross over the lesion T. Therefore, the procedure can be completed without the overall device coming into contact with the lesion T.

### Second Embodiment

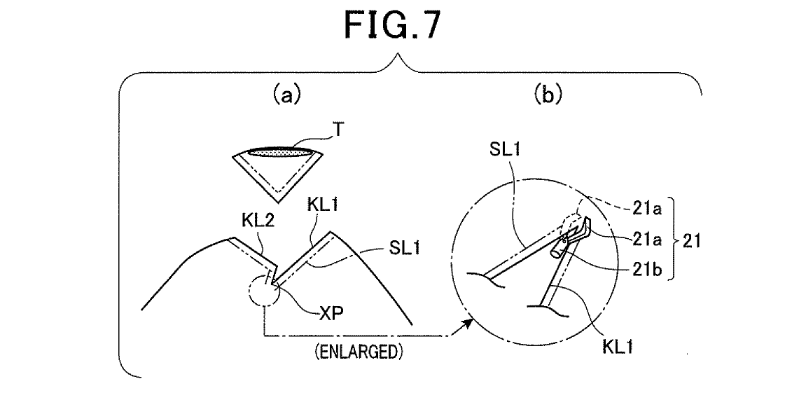

Next, an operative method according to a second embodiment will be described with reference to FIG. 7.

FIG. 7 shows a state in which a tissue piece including the lesion T is excised by the linear stapler 120 (FIG. 7(a)). In the example shown in FIG. 7, of the ends of the cut edge KL1 formed by the first stapling procedure, an end XP (referred to, hereinafter, as an “intersecting end”) that intersects with the cut edge KL2 formed by another (second) stapling procedure is formed projecting towards the gastrointestinal tract tissue side. In this instance, the folded lumen wall that is located further inward than the intersecting end XP is sandwiched and fastened by a clip 21 from both sides in the thickness direction, such as to seal the intersecting end XP. The clip 21 shown in FIG. 7 has a pair of clamping sections 21a and a connecting section 21b connected to the pair of clamping sections 21a. The connecting section 21b generates a clamping force in the pair of clamping sections 21a by elastic force or the like. The clip 21 is a known clip. As a result, leakage and dissemination into the abdominal cavity from a miniscule through-hole formed by the intersecting end XP can be more completely prevented.

The operation for fastening the clip 21 may be performed when the check for through-holes is performed at Step S112 of the flowchart shown in FIG. 3 and a leak is found. Alternatively, the operation may be performed before the check is performed.

### Third Embodiment

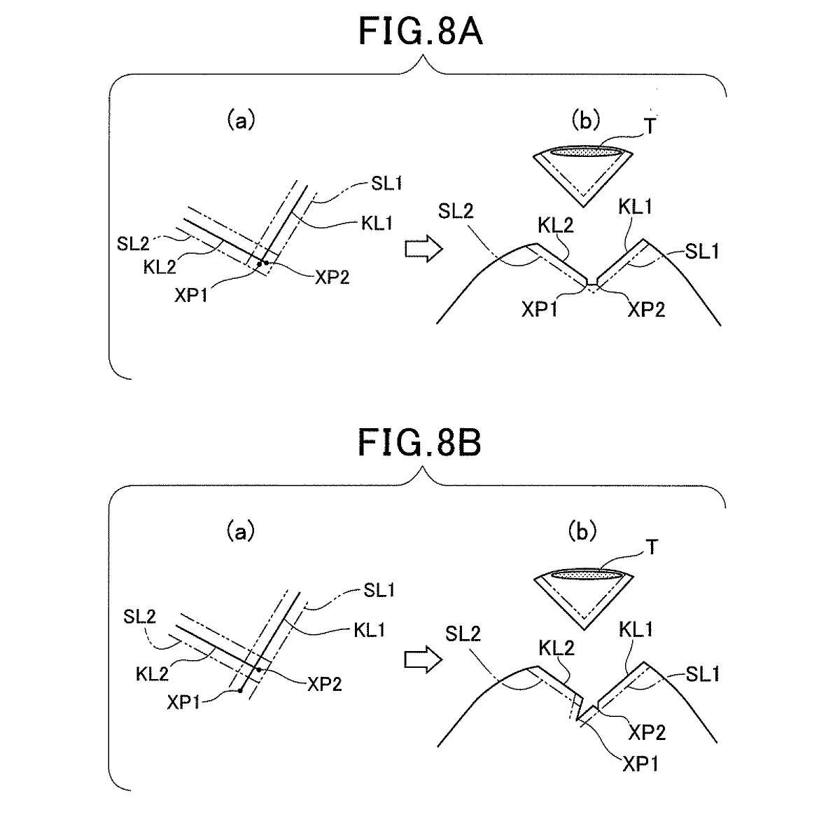

The method for sealing a through-hole formed in the gastrointestinal tract wall by the intersecting end XP is not limited to that shown in FIG. 7. A method shown in FIG. 8A may also be used. FIG. 8A (a) is a schematic diagram of an example of a method for forming the cut edges and joined sections. FIG. 8A (b) is a diagram of the lesion T being excised by the linear stapler 120 in an instance in which the method shown in FIG. 8A (a). In a manner similar to FIG. 6K, FIG. 8A (b) shows a state in which the tissue is folded such that the inner surface of the gastrointestinal tract faces outward and the outer surface of the gastrointestinal tract faces inward.

For example, as shown in FIG. 8A, when the cut edges KL1 and KL2 are formed to intersect with each other, the steps of joining and cutting are performed such that each intersecting end XP1, XP2 of each cut edge KL1, KL2 is positioned further towards the respective other intersecting cut edge KL2, KL1 than the joined section SL2, SL1 formed along the respective other intersecting cut edge KL2, KL1. In other words, in the example in FIG. 8A, when the joining and cutting procedures are performed more than once, the latter joining and cutting procedure is performed such that the intersecting end XP2 formed by the latter joining and cutting procedure is positioned further towards the cut edge KL1 formed by the former joining and cutting procedure than the joined section SL1 formed by the former joining and cutting procedure. In addition, the latter joining and cutting procedure is performed such that the intersecting end XP1 formed by the former joining and cutting procedure is positioned further towards the cut edge KL2 formed by the latter joining and cutting procedure than the joined section SL2 formed by the latter joining and cutting procedure. In the example in FIG. 8A, the cut surface is formed on one side of the joined sections al and SL2 that intersect with each other, without crossing over the joined sections SL1 and SL2. Therefore, the ends of the cut edges KL2 and KL1 are sealed by the joined sections SL1 and SL2 formed in the intersecting direction. Communication of fluid in and out of the gastrointestinal tract can be prevented.

On the other hand, FIG. 8B is a diagram of a joining and cutting state that is not according to the third embodiment. In FIG. 8B (a), only the cut edges KL1 and KL2 and the joined sections SL1 and SL2 are shown for convenience of description. The schematic diagram in FIG. 8B (b) shows the state of the gastrointestinal tract tissue when a stapling method such as this is used. In this example, the intersecting end XP2 formed by the latter stapling procedure is positioned between the joined section SL1 and the cut edge KL1 of the former stapling procedure. However, the intersecting end XP1 formed by the former stapling procedure crosses over the joined section SL2 formed by the latter stapling procedure, and reaches the area on the opposite side of the cut edge KL2. In this instance, as shown in FIG. 8B (b), the former intersecting end XP1 is not sealed by the joined section SL2, and fluid communicates between the inside of the gastrointestinal tract and the abdominal cavity. In such instances, the intersecting end XP1 can be sealed using the clip 21, as according to the second embodiment.

As described above, the joined sections SL1 and SL2 formed during the joining and cutting procedures can be used to seal the intersecting ends XP2 and XP1 of the respective intersecting cut edge KL2 or KL1. As a result, occurrence of fluid communication due to the intersecting ends XP1 and XP2 can be efficiently prevented.

### Fourth Embodiment

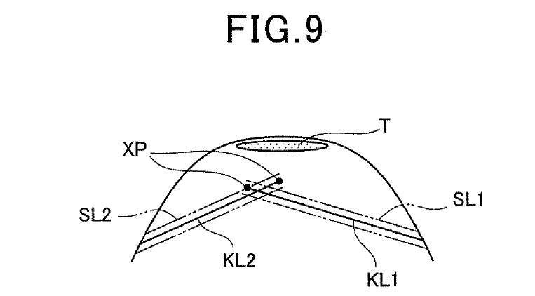

In addition, an excision method shown in FIG. 9 can be given as a method for preventing a through-hole from being formed in the gastrointestinal tract wall by the intersecting end XP. FIG. 9 is a diagram of the lesion T being excised by the linear stapler 120. In a manner similar to FIG. 6G to FIG. 6K and so on, FIG. 9 shows a state in which the tissue is folded such that the inner surface of the gastrointestinal tract faces outward and the outer surface of the gastrointestinal tract faces inward. As shown in FIG. 9, according to a fourth embodiment, each joining and cutting procedure is performed such as to form the intersecting end XP on the excised piece side including the lesion T.

### Fifth Embodiment

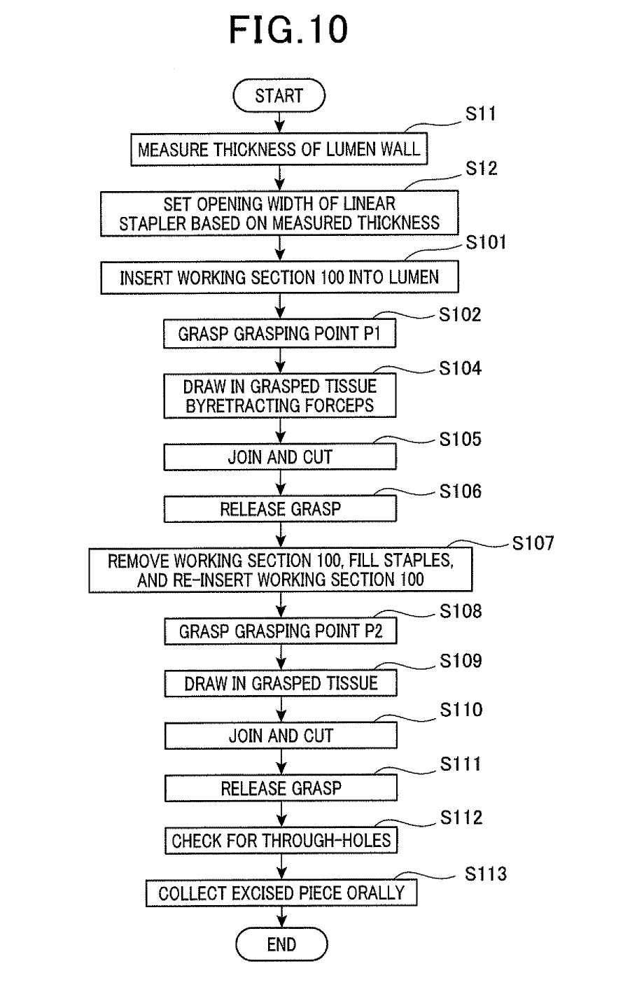

A fifth embodiment will be described with reference to FIG. 10. The operative method according to the fifth embodiment is basically similar to that according to the first embodiment. However, preparatory steps S11 and S12 are provided before processing of the lesion T is started by the working section 100.

Specifically, at Step S11, the thickness of the gastrointestinal tract wall to be excised is measured. Measurement is performed, for example, using endoscopic ultrasound and calculated from an endoscopic ultrasound image. Next, the opening width H (see FIG. 6B) of the linear stapler 120 is set based on the measurement result of the thickness at Step S11. For example, the setting may be performed by an appropriate linear stapler 120 being selected from linear staplers 120 having different opening widths. Alternatively, a mechanism for adjusting the opening width may be provided in the linear stapler 120. Moreover, staples 126 having an appropriate size may be selected. The opening width of the linear stapler 120 is set to a size preventing organs and tissue outside of the gastrointestinal tract from entering, such that the organs and tissue outside of the gastrointestinal tract are not caught between the gastrointestinal tract wall and drawn in when the gastrointestinal tract wall is drawn into the linear stapler 120. For example, the opening width is set to be equal to or less than the thickness when the gastrointestinal tract wall is folded.

Subsequent Step S101 to Step S113 are similar to those according to the first embodiment. Processing of the lesion T is performed using the linear stapler 120 that is set to a predetermined opening width at the above-described steps. As a result, when the gastrointestinal tract wall is drawn into the linear stapler 120, the organs and tissue outside of the gastrointestinal tract can be prevented from being drawn in, severed, and the like with the gastrointestinal tract wall.

### Variation Example

Embodiments of the present invention are described above. However, application of the present invention is not limited to the above-described examples.

For example, according to the above-described embodiments, an instance is described of excision of the stomach wall. However, the present invention can be applied to excision of tissue having a lumen, such as blood vessels, the esophagus, the duodenum, and the colon.

In addition, the present invention is not limited to that in which the grasping section 130 is drawn in along the formation direction of the joined section and the cut edge by the linear stapler 120 as according to the present embodiments. The direction in which the grasping section 130 is drawn in may intersect with the joined section and the cut edge formed by the linear stapler 120.

In addition, the direction in which the grasping section 130 is drawn in and the formation direction of the joined section and the cut edge by the linear stapler 120 may differ from the axial direction of the insertion section 15.

Furthermore, according to the above-described embodiments, an instance is described in which the stapling (and accompanying cutting) procedure is performed twice. However, the stapling procedure may be performed three times or more. The shape of the excised piece can be changed in adherence to the shape of the lesion T. In addition, according to the above-described embodiment, an angle is formed between differing cut edges. However, the cut edges may form a single, straight line. In addition, the length of the joined section is not necessarily required to be the same in each stapling procedure, and can be changed within the length of the cutting section 128.

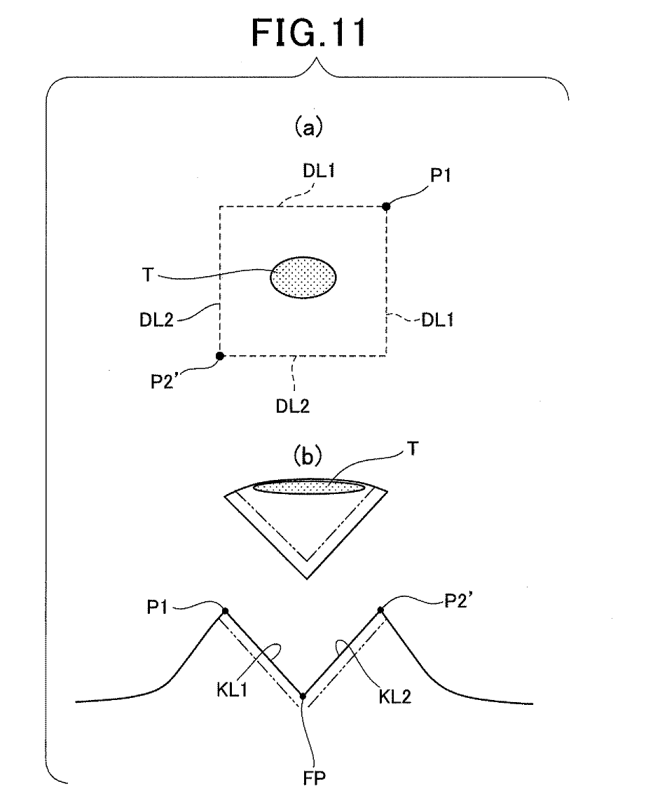

Moreover, the present invention is not limited to an instance in which cutting is performed such that a distal end FP of the cut edge formed by a preceding stapling (and accompanying cutting) procedure, or in other words, the area cut by the distal end of the cutting section 128, intersects with the proximal end of the cutting section 128 in the subsequent stapling procedure, as according to the first embodiment. For example, in the example according to the first embodiment, the distal ends FP of the cut edges may intersect with each other, as shown in FIG. 11. FIG. 11 (a) shows a schematic diagram of a relationship between a lesion and a procedure site in this case, corresponding to FIG. 5. FIG. 11 (b) is a schematic diagram showing the gastrointestinal tract and the excised piece including lesion T after this procedure.

What is claimed is:

1. An operative method for performing full-thickness excision of tissue of a lesion in a lumen through a natural orifice, the operative method comprising:
a folding step of folding a lumen wall of an excision site such that an outer surface of the lumen wall faces inward, while pushing external organs outside of the lumen from inside of the lumen in a direction away from the excision site, such as to exclude the external organs outside of the lumen from the excision site; and
a joining and cutting step of joining and cutting a site where processing at the folding step is performed, using a stapler including a cutting member, and forming a joined section in which layers of the folded tissue are joined and a cut surface of the tissue in a folded state, wherein
the lesion is excised by the joining and cutting step being repeatedly performed along the periphery of the legion.

2. The operative method according to claim 1, wherein:
the folding step is performed using the stapler, by pushing the organs outside of the lumen in the direction away from the lesion by the stapler.

3. The operative method according to claim 1, wherein:
at the folding step, the organs outside of the lumen are pushed along a formation direction of the cut surface formed along the periphery of the lesion at the joining and cutting step.

4. The operative method according to claim 1, wherein:
processing at the folding step is performed on lumen tissue pulled by a pulling member along a formation direction of the cut surface formed along the periphery of the lesion at the joining and cutting step.

5. The operative method according to claim 1, wherein:
in repeated joining and cutting steps, cut surfaces that are continuous along the periphery of the lesion are formed, and
the operative method further includes a sealing step for sealing the lumen to prevent communication of fluid inside and outside of the lumen through an intersecting end formed in the lumen and not configuring an end of the excision site, among ends in a formation direction of the cut surfaces formed at repeated joining and cutting steps.

6. The operative method according to claim 5, wherein:
in the sealing step, the intersecting end is sandwiched and fastened by a clip from both sides of the lumen wall.

7. The operative method according to claim 5, wherein:
in repeated joining and cutting steps, cut surfaces are formed that are continuous along the periphery of the lesion, and joined sections are formed along the formation direction of the cut surfaces in at least a remaining-side lumen portion that remains after the lesion is excised, and
when the cut surfaces formed by repeated joining and cutting steps intersect, the sealing step is performed by performing the joining and cutting steps such that the intersecting end of the cut surface formed by the joining and cutting step is formed further towards the respective intersecting cut surface than the joined section formed in the remaining-side lumen portion along the formation direction of the respective intersecting cut surface.

8. The operative method according to claim 1, wherein:
in the joining and cutting step, joining and cutting is performed by leading the excision site into an opening communicating with a procedure space of the stapler; and
in the folding step, the excision site is led into the opening while pushing the external organs from inside of the lumen by the stapler; and
the operative method further includes a measuring step for measuring the thickness of the lumen wall, before the folding step, and the joining and cutting step, and
a setting step for setting an opening width of the stapler based on the measurement result at the measuring step.

9. A operative method for performing full-thickness excision of tissue of a lesion in a lumen through a natural orifice, the operative method including a joining and cutting step of joining and cutting the periphery of the lesion using a stapler including a cutting member, and forming a joined section where layers of the folded tissue are joined and a cut surface of the tissue in a folded state, in which the lesion is excised by the joining and cutting steps being repeatedly performed along the periphery of the lesion, the operative method wherein:
in each repeated joining and cutting step, joining and cutting is performed such that an intersecting end that does not configure an end of the excision site, among ends in a formation direction of the cut surfaces formed along the periphery of the lesion, is positioned towards the excision site side including the lesion to be excised.
